(19) United States
(12) Patent Application Publication
(10) Pub. No.: US 2011/0313199 A1
(43) Pub. Date: Dec. 22, 2011

(54) PROCESSES FOR PREPARING SUBSTANTIALLY PURE ARFORMOTEROL AND ITS INTERMEDIATES

(75) Inventors: Girish Dixit, Uttar Pradesh (IN); Nandkumar Gaikwad, Maharashtra (IN); Nitin Sharadchandra Pradhan, Maharashtra (IN)

(73) Assignee: ACTAVIS GROUP PTC EHF, Hafnarfjordur (IS)

(21) Appl. No.: 13/141,167

(22) PCT Filed: Dec. 28, 2009
(86) PCT No.: PCT/IB09/08097

§ 371 Date: Sep. 2, 2011

(30) Foreign Application Priority Data
Dec. 26, 2008 (IN) 3281/CHE/2008
Mar. 13, 2009 (IN) 575/CHE/2009

(51) Int. Cl. C07C 233/88; C07B 57/00; C07C 213/02; C07C 209/28; C07C 205/37; C07C 205/38

(52) U.S. Cl. 564/221; 568/587; 568/586; 564/418; 564/415; 564/304

(57) ABSTRACT

Provided herein are improved, convenient and industrially advantageous processes for the preparation of N-[2-hydroxy-5-[(1R)-1-hydroxy-2-[[(1R)-2-(4-methoxyphenyl)-1-methylethyl]amino]ethyl]phenyl]formamide (Arformoterol) or a pharmaceutically acceptable salt thereof, in high yield and purity. Provided further herein is an improved and industrially advantageous process for the preparation of a substantially enantiomerically pure arformoterol intermediate, (R)-4-methoxy-α-methyl-N-(phenylmethyl)benzeneethanamine.

### CROSS REFERENCE TO RELATED APPLICATION

This application claims the benefit of priority to Indian provisional application Nos. 3281/CHE/2008, filed on Dec. 26, 2008; and 575/CHE/2009, filed on Mar. 13, 2009; which are incorporated herein by reference in their entirety.

### FIELD OF THE DISCLOSURE

The present disclosure relates to improved and industrially advantageous processes for the preparation of N-[2-hydroxy-5-[(1R)-1-hydroxy-2-[[(1R)-2-(4-methoxyphenyl)-1-methyl ethyl]amino]ethyl]phenyl]formamide (Arformoterol), or a pharmaceutically acceptable salt thereof, and its intermediates, in high yield and purity.

### BACKGROUND

U.S. Pat. No. 3,994,974 discloses a variety of α-aminomethylbenzyl alcohol derivatives, processes for their preparation, pharmaceutical compositions comprising the derivatives, and method of use thereof. These compounds have the utility as β-adrenergic stimulants and thus have great activity on respiratory smooth muscle and are suitable as bronchodilating agents. Among them, Formoterol, (±)-N-[2-hydroxy-5-[1-hydroxy-2-[[2-(4-methoxyphenyl)-1-methylethyl]amino]ethyl]phenyl]formamide, is a highly potent and β₂-selectiveadrenoceptor agonist having a long lasting bronchodilating effect when inhaled. Formoterol is represented by the following structural formula:

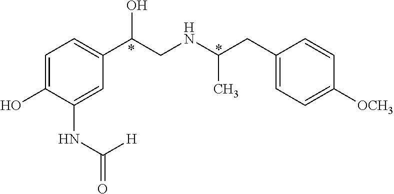

Formoterol has two chiral centers in the molecule, each of which can exist in two possible configurations. This gives rise to four combinations: (R,R), (S,S), (R,S) and (S,R). (R,R) and (S,S) are minor images of each other and are therefore enantiomers; (R,S) and (S,R) are similarly an enantiomeric pair. The mirror images of (R,R) and (S,S) are not, however, superimposable on (R,S) and (S,R), which are diastereomers. The order of potency of the isomers is (R,R)>>(R,S)═(S,R)>(S,S), and the (R,R)-isomer is 1000-fold more potent than the (S,S)-isomer. Administration of the pure (R,R)-isomer also offers an improved therapeutic ratio.

Various processes for the preparation of formoterol, its enantiomers and related compounds, and their pharmaceutically acceptable salts are disclosed in U.S. Pat. Nos. 3,994,974; 5,434,304; 6,268,533 and 6,472,563; Chem. Pharm. Bull. 26, 1123-1129 (1978); Chirality 3, 443-450 (1991); Drugs of the Future 2006, 31(11), 944-952; and PCT Publication No. WO 2008/035380A2.

The syntheses of all four isomers of formoterol have been reported in the journals, Chem. Pharm. Bull. 26, 1123-1129 (1978) (hereinafter referred to as the ‘CPB Journal’), and Chirality 3, 443-450 (1991) (hereinafter referred to as the ‘Chirality journal’). In the CPB Journal, the (R,R)- and (S,S)-isomers are obtained by diastereomeric crystallization of racemic formoterol with tartaric acid. In the Chirality journal, racemic 4-benzyloxy-3-nitrostyrene oxide is coupled with an optically pure (R,R)- or (S,S)—N-(1-phenylethyl)-N-(1-(p-methoxyphenyl)-2-propyl)amine to give a diastereomeric mixture of formoterol precursors, which are then separated by semipreparative HPLC and transformed to the pure formoterol isomers. Both syntheses suffer long synthetic procedure and low overall yield and are impractical for large scale production of optically pure (R,R)- or (S,S)-formoterol.

U.S. Pat. No. 6,268,533 discloses that the L-(+)-tartrate salt of R,R-formoterol is unexpectedly superior to other salts of R,R-formoterol (arformoterol), being easy to handle, pharmaceutically innocuous and non-hygroscopic. Arformoterol, N-[2-hydroxy-5-[(1R)-1-hydroxy-2-[[(1R)-2-(4-methoxyphenyl)-1-methylethyl]amino]ethyl]phenyl]formamide, is a highly potent and selective β₂-adrenergic bronchodilator. Arformoterol is represented by the following structural formula 1:

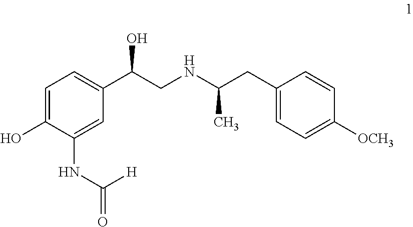

As per the process described in the U.S. Pat. No. 6,268,533, arformoterol tartrate is prepared by enantioselective reduction of 2-bromo-4′-benzyloxy-3′-nitroacetophenone with borane methyl sulfide in the presence of a chiral oxazaborolidine to produce R-α-(bromomethyl)-4-phenylmethoxy-3-nitrobenzenemethanol, which is then hydrogenated in a Parr hydrogenator in the presence of platinum oxide catalyst to afford the corresponding amino compound, followed by formylation reaction with formic acid in the presence of acetic anhydride to produce (R)—N-[5-(2-bromo-1-hydroxyethyl)-2-(phenylmethoxy)phenyl]formamide, which is then treated with potassium carbonate to produce (R)—N-[5-oxiranyl-2-(phenylmethoxy)phenyl]formamide. The epoxide compound is then condensed with (R)-4-methoxy-α-methyl-N-(phenylmethyl)benzene ethanamine L-mandelate to produce a dibenzyl protected compound, which is then hydrogenated in the presence of palladium on carbon catalyst to produce arformoterol followed by reaction with L-tartaric acid to produce arformoterol tartrate.

Five different synthetic routes for preparing arformoterol and its tartrate salt have been reported in the Drugs of the Future 2006, 31(11) (hereinafter referred to as the ‘DOF article’). According to first synthetic process, arformoterol tartrate is prepared by resolution of racemic formoterol (I) with D-(+)-tartaric acid. Analogously, racemic p-methoxy-α-methylphenethylamine (II) is resolved by means of D-(+)-tartaric acid to provide the (R)-amine compound (III), which is used as the starting material for the asymmetric synthesis of arformoterol tartrate.

According to second synthetic process as reported in the DOF article, arformoterol is prepared by condensation of (p-methoxyphenyl)acetone (IV) with 1(R)-phenylethylamine (V), followed by diastereoselective hydrogenation of the intermediate imine over Raney nickel to produce the (R,R)-amine (VI), which is then reacted with the racemic epoxide (VII) to produce the amino alcohol adduct (VIII) as an epimeric mixture, followed by subsequent nitro group reduction and formylation in the presence of formic acid and Raney nickel to produce the corresponding mixture of epimeric formamides (IX) and (X), which are separated utilizing semi-preparative chromatography. The desired isomer (X) is finally deprotected by hydrogenation over Pd/C.

According to third synthetic process as reported in the DOF article, arformoterol is prepared by condensation of (R)-4-benzyloxy-3-nitro-styrenoxide (XVII) with (R)-4-methoxy-α-methyl-N-(phenylmethyl)benzene ethanamine (XII) to produce the desired (R,R)-amino alcohol (XVIII) followed by nitro group reduction and subsequent formylation of the resulting amine (XIX) to produce the formamide compound (XX), which is then converted to arformoterol by catalytic hydrogenolysis of its benzyl protecting groups. The amine compound (XII) is prepared by reductive amination of (p-methoxyphenyl)acetone (IV) with benzylamine using H₂ and Pt/C to produce the racemic secondary amine (XI), which is resolved using (S)-mandelic acid to give the optically pure (R)-amine (XII). The (R)-4-benzyloxy-3-nitro-styrenoxide (XVII) is in turn prepared by enantioselective reduction of 2-bromo-4′-benzyloxy-3′-nitroacetophenone (XIII) with borane in the presence of the chiral oxazaborolidines (XVa,b) to give the (R)-bromohydrin (XVI), which is converted to the epoxide (XVII) in the presence of an aqueous base.

The fourth synthetic process as reported in the DOF article, involves a chemoenzymatic approach.

According to fifth synthetic process as reported in the DOF article, arformoterol is prepared by reduction of the enantiopure bromohydrin (XVI) at the nitro group by catalytic hydrogenation. The resulting amine (XXXII) is then converted to formamide (XXXIII) followed by condensation with an amine compound (XII) to produce the amino alcohol (XX), which is finally deprotected by hydrogenation over Pd/C to produce arformoterol.

Arformoterol obtained by the process described in the aforementioned prior art does not have satisfactory purity. Unacceptable amounts of impurities are formed along with arformoterol. The yield of arformoterol obtained is also poor and the processes involve column chromatographic purifications. Methods involving column chromatographic purifications are generally undesirable for large-scale operations, thereby making the process commercially unfeasible.

However, the prior art methods for preparing arformoterol have the following disadvantages and limitations:
i) the reduction of 2-bromo-4′-benzyloxy-3′-nitroacetophenone to produce the (R)-2-bromo-1-[(4-benzyloxy)-3-nitrophenyl]ethanol involves the use of twin reagents i.e., the use of reducing agent borane dimethylsulfide in the presence of an expensive chiral catalyst such as a chiral oxazaborolidine;
ii) the use of additional, expensive, hazardous and difficult to handle reagents such as borane dimethylsulfide in the presence of chiral oxazaborolidines, which are not advisable for scale up operations;
iii) the reduction of the compounds at nitro group to produce the corresponding amine compounds involves the use of expensive, pyrophoric, corrosive and explosive reagents like Raney-Ni, H₂/PtO₂ and Fe/HCl;
iv) since Raney Ni is a pyrophoric and explosive reagent, the use of HCl in the processes corrodes the equipment used in the manufacture, and the PtO₂ is a highly explosive and expensive reagent, and hence the use of these reagents is not advisable for scale up operations.

In the preparation of arformoterol, (R)-4-methoxy-α-methyl-N-(phenylmethyl)benzene ethanamine of formula 5a(i):

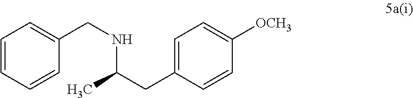

is a key intermediate. According to U.S. Pat. Nos. 5,965,622 and 6,268,533, the benzylamine compound of formula 5a(i) is prepared by the reaction of 4-methoxyphenylacetone with N-benzylamine in methanol to produce a reaction mass containing the imine compound, which is in situ hydrogenated in the presence of a 5% platinum on carbon catalyst to give racemic 4-methoxy-α-methyl-N-(phenylmethyl)benzene ethanamine, which is then resolved using L-mandelic acid in an alcohol solvent such as methanol. The optically pure benzylamine mandelic salt is obtained after four or five crystallizations, and is then treated with a base such as aqueous sodium hydroxide, aqueous sodium carbonate or aqueous ammonia in the presence of an inert organic solvent such as t-butyl methyl ether or ethyl acetate, followed by evaporation of the solvent to produce (R)-4-methoxy-α-methyl-N-(phenylmethyl)benzene ethanamine.

The process for the preparation of (R)-4-methoxy-α-methyl-N-(phenylmethyl)benzene ethanamine of formula 5a(i) disclosed in the U.S. Pat. Nos. 5,965,622 and 6,268,533 suffers from disadvantages such as probable racemization, high cost of reagents, low yields of the product, extra purifications steps to obtain the final product, and repeated crystallizations. The process is not advisable for scale up operations.

Based on the aforementioned drawbacks, the prior art processes may be unsuitable for the preparation of arformoterol in commercial scale operations.

A need remains for an improved and commercially viable process of preparing a substantially pure arformoterol or a pharmaceutically acceptable salt thereof, preferably arformoterol tartrate, to resolve the problems associated with the processes described in the prior art, and that will be suitable for large-scale preparation. Desirable process properties include less hazardous, environmentally friendly and easy to handle reagents, reduced cost, greater simplicity, increased purity, and increased yield of the product, thereby enabling the production of arformoterol, its intermediates and its pharmaceutically acceptable acid addition salts in high purity and in high yield.

### SUMMARY

In one aspect, provided herein are efficient, industrially advantageous and environmentally friendly processes for the preparation of N-[2-hydroxy-5-[(1R)-1-hydroxy-2-[[(1R)-2-(4-methoxyphenyl)-1-methylethyl]amino]ethyl]phenyl]formamide of formula 1 (Arformoterol) or a pharmaceutically acceptable salt thereof in high yield and with high chemical and enantiomeric purity. Moreover, the reagents used in the processes disclosed herein are non-hazardous and easy to handle at a commercial scale and also allow reduced reaction times. The processes avoid the tedious and cumbersome procedures of the prior processes and are convenient to operate on a commercial scale.

In another aspect, provided herein is an efficient, industrially advantageous and environmentally friendly process for the preparation of enantiomerically pure arformoterol key starting material, (R)-α-(bromomethyl)-4-phenylmethoxy-3-nitrobenzenemethanol, in high yield and purity, by enantioselective reduction of 2-bromo-4′-benzyloxy-3′-nitroacetophenone with (−)-β-chlorodiisopinocampheylborane (also known as ‘(−)-DIP chloride’).

In another aspect, provided herein is an efficient, industrially advantageous and environmentally friendly process for the preparation of stereochemically highly pure arformoterol intermediate, (R,R)-3-amino-α-[[[2-(4-methoxyphenyl)-1-methylethyl](phenyl methyl)amino]methyl]-4-(phenylmethoxy)-benzenemethanol, in high yield and purity, comprising reducing (R,R)-3-nitro-α-[[[2-(4-methoxyphenyl)-1-methylethyl](phenylmethyl)amino]methyl]-4-(phenylmethoxy)-benzenemethanol with sodium dithionite.

In another aspect, provided herein is an efficient, commercially viable and environmentally friendly process for the preparation of enantiomerically pure arformoterol intermediate, (R)-4-methoxy-α-methyl-N-(substituted or unsubstituted phenylmethyl)benzene ethanamine of formula 5a. Advantageously, the reagents used for present invention are less hazardous, easy to handle at commercial scale, and less expensive than those used in other processes.

In still another aspect, encompassed herein is the use of enantiomerically pure (R)-α-(bromomethyl)-4-phenylmethoxy-3-nitrobenzenemethanol obtained by the process disclosed herein for preparing arformoterol.

In yet another aspect, encompassed herein is the use of stereochemically pure (R,R)-3-amino-α-[[[2-(4-methoxyphenyl)-1-methylethyl](phenylmethyl)amino]methyl]-4-(phenyl methoxy)-benzenemethanol obtained by the process disclosed herein for preparing arformoterol.

In still another aspect, encompassed also herein is the use of enantiomerically pure (R)-4-methoxy-α-methyl-N-(substituted or unsubstituted phenylmethyl)benzene ethanamine of formula 5a obtained by the process disclosed herein for preparing arformoterol.

### DETAILED DESCRIPTION

According to one aspect, there is provided a process for the preparation of arformoterol of formula 1:

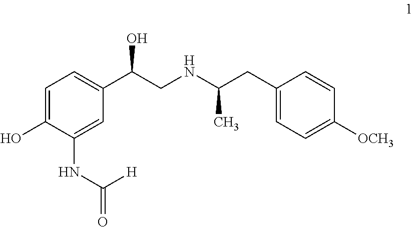

or a pharmaceutically acceptable salt thereof; comprising:
a) reducing an acetophenone compound of formula 8:

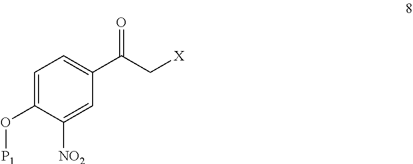

wherein ‘X’ represents a halogen atom, selected from the group consisting of F, Cl, Br and I; and ‘P₁’ is a hydroxy-protecting group;
with (−)-β-chlorodiisopinocampheylborane to produce an enantiomerically pure (R)-halohydrin compound of formula 7:

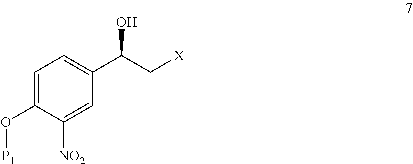

wherein ‘X’ and ‘P₁’ are as defined in formula 8;
b) treating the (R)-halohydrin compound of formula 7 with a base to produce an enantiomerically pure oxirane compound of formula 6:

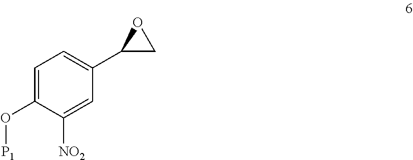

wherein ‘P₁’ is as defined in formula 8;
c) condensing the oxirane compound of formula 6 with an enantiomerically pure (R)-amine compound of formula 5:

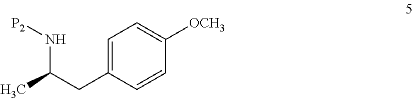

wherein ‘P₂’ is an amine-protecting group; to produce an (R,R)-nitro alcohol compound of formula 4:

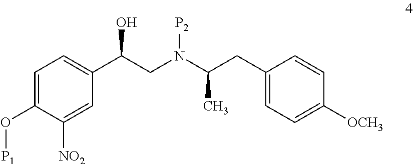

wherein the ‘P₁’ and ‘P₂’ are as defined in formulae 8 and 5;
d) reducing the (R,R)-nitro alcohol compound of formula 4 with sodium dithionite in a solvent to produce an (R,R)-amino alcohol compound of formula 3:

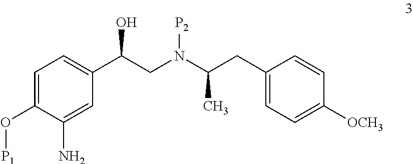

wherein the ‘P₁’ and ‘P₂’ are as defined above;
e) formylating the compound of formula 3 with formic acid in the presence of polyethylene glycol to produce an (R,R)-formamide compound of formula 2:

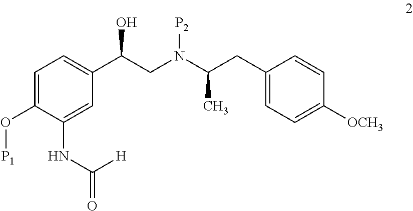

wherein the ‘P₁’ and ‘P₂’ are as defined above;
f) deprotecting the (R,R)-formamide compound of formula 2 to produce the substantially pure arformoterol of formula 1, and optionally converting the arformoterol formed into a pharmaceutically acceptable acid addition salt thereof.

In one embodiment, the halogen atom ‘X’ in the compounds of formulae 7 and 8 is Cl or Br, and more specifically, X is Br.

The hydroxy-protecting group ‘P₁’ in the compounds of formulae 2, 3, 4, 6, 7 and 8; and the amine-protecting group ‘P₂’ in the compounds of formulae 2, 3, 4 and 5, are any known such groups, for example as described in the relevant chapters of standard reference works such as J. F. W. McOmie, “Protective Groups in Organic Chemistry”, Plenum Press, London and New York 1973, in T. W. Greene and P. G. M. Wuts, “Protective Groups in Organic Synthesis”, Third edition, Wiley, N.Y. 1999, in “The Peptides”; Volume 3 (editors: E. Gross and J. Meienhofer), Academic Press, London and New York 1981.

Exemplary hydroxy-protecting groups ‘P₁’ include, but are not limited to, aryl- or aryloxy-substituted methyl groups such as benzyl, diphenylmethyl, triphenylmethyl and benzyloxymethyl. A specific hydroxy-protecting group ‘P₁’ is benzyl.

Exemplary amine-protecting groups ‘P₂’ include, but are not limited to, benzyl and substituted benzyl groups.

The term “substituted benzyl” as used herein refers to an amine protecting group that contains the benzyl (or phenylmethyl) nucleus substituted with one or more substituents that do not interfere with its function as a protecting group. Exemplary substituents include, but are not limited to, C₁ to C₆-alkyl, C₁ to C₆-alkoxyl, halogen and combinations thereof. A specific amine protecting group ‘P₂’ is benzyl.

The term “substantially pure arformoterol or a pharmaceutically acceptable salt thereof” refers to the arformoterol or a pharmaceutically acceptable salt thereof having total purity of greater than about 98%, specifically greater than about 99%, more specifically greater than about 99.5%, and most specifically greater than about 99.9% (measured by HPLC).

In one embodiment, the reduction in step-(a) is carried out in a solvent selected from the group consisting of an alcohol, a chlorinated hydrocarbon, a hydrocarbon, a nitrile, esters, an ether, a polar aprotic solvent, and mixtures thereof.

Specifically, the solvent used in step-(a) is selected from the group consisting of methanol, ethanol, n-propanol, isopropanol, n-butanol, isobutanol, tert-butanol, amyl alcohol, hexanol, acetonitrile, ethyl acetate, methyl acetate, isopropyl acetate, tert-butyl methyl acetate, ethyl formate, dichloromethane, ethylene dichloride, chloroform, carbon tetrachloride, tetrahydrofuran, dioxane, diethyl ether, diisopropyl ether, monoglyme, diglyme, n-pentane, n-hexane, n-heptane, cyclohexane, toluene, xylene, N,N-dimethylformamide, N,N-dimethylacetamide, dimethylsulfoxide, and mixtures thereof; more specifically the solvent is selected from the group consisting of dichloromethane, toluene, diisopropyl ether, hexane, and mixtures thereof; and a most specific solvent is dichloromethane.

In one embodiment, the (−)-β-chlorodiisopinocampheylborane is used in a molar ratio of about 0.5 to 2.5 moles, specifically about 1 to 2 moles, per 1 mole of the acetophenone compound of formula 8 in order to ensure a proper course of the reaction.

In another embodiment, the reaction in step-(a) is carried out at a temperature of below about 50° C. for at least 30 minutes, specifically at a temperature of about −25° C. to about 40° C. for about 1 hour to about 8 hours, and more specifically at a temperature of about 0° C. to about 30° C. for about 2 hours to about 7 hours. In another embodiment, the reaction mass may be quenched with a solution of aqueous base after completion of the reaction.

The reaction mass containing the (R)-halohydrin compound of formula 7 obtained in step-(a) may be subjected to usual work up such as a washing, a filtration, an extraction, an evaporation or a combination thereof. The reaction mass may be used directly in the next step to produce the oxirane compound of formula 6, or the compound of formula 7 may be isolated and then used in the next step.

In one embodiment, the (R)-halohydrin compound of formula 7 formed in step-(a) is isolated as a solid from a suitable organic solvent by methods such as cooling, seeding, partial removal of the solvent from the solution, by adding an anti-solvent to the solution, evaporation, vacuum drying, spray drying, freeze drying, or a combination thereof.

In another embodiment, the organic solvent used to isolate the (R)-halohydrin compound of formula 7 is an aliphatic or aromatic hydrocarbon solvent selected from the group consisting of heptane, pentane, hexane, toluene, xylene, cyclohexane, petroleum ether, and mixtures thereof. Specific organic solvents are hexane, toluene, and mixtures thereof.

In one embodiment, a specific (R)-halohydrin compound of formula 7 prepared by the process described herein is (R)-α-(bromomethyl)-4-phenylmethoxy-3-nitrobenzenemethanol of formula 7a (formula 7, wherein P₁ is benzyl, and X is Br):

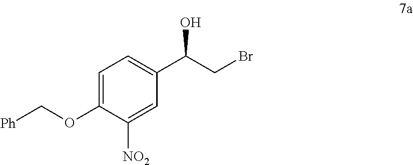

In another embodiment, the base used in step-(b) is an inorganic base. Exemplary inorganic bases include, but are not limited to, ammonia; hydroxides, alkoxides, carbonates and bicarbonates of alkali or alkaline earth metals. Specific inorganic bases are ammonia, sodium hydroxide, calcium hydroxide, magnesium hydroxide, potassium hydroxide, lithium hydroxide, sodium carbonate, potassium carbonate, lithium carbonate, sodium bicarbonate, potassium bicarbonate, sodium tert-butoxide, sodium isopropoxide, potassium tert-butoxide, and mixtures thereof; and more specifically aqueous ammonia, sodium hydroxide, potassium hydroxide, sodium bicarbonate, sodium carbonate and potassium carbonate.

In one embodiment, the reaction in step-(b) is carried out in a solvent selected from the group consisting of water, an alcohol, a ketone, a nitrile, an ether, a polar aprotic solvent, and mixtures thereof. Specifically, the solvent is selected from the group consisting of water, methanol, ethanol, n-propanol, isopropanol, n-butanol, isobutanol, tert-butanol, amyl alcohol, hexanol, acetone, methyl ethyl ketone, methyl isobutyl ketone, methyl tert-butyl ketone, acetonitrile, tetrahydrofuran, dioxane, diethyl ether, diisopropyl ether, monoglyme, diglyme, N,N-dimethylformamide, N,N-dimethylacetamide, dimethylsulfoxide, and mixtures thereof;

and more specifically the solvent is selected from the group consisting of water, tetrahydrofuran, acetone, methanol, ethanol, n-propanol, isopropanol, n-butanol, isobutanol, tert-butanol, amyl alcohol, and mixtures thereof.

In another embodiment, the reaction in step-(b) is carried out at a temperature of about 0° C. to about 100° C. for at least 30 minutes, specifically at a temperature of about 0° C. to about 50° C. for about 1 hour to about 6 hours, and most specifically at a temperature of about 0° C. to about 35° C. for about 2 hours to about 5 hours. In another embodiment, the reaction mass may be quenched with water after completion of the reaction.

The reaction mass containing the oxirane compound of formula 6 obtained in step-(b) may be subjected to usual work up methods as described above.

In another embodiment, the oxirane compound of formula 6 formed in step-(b) is isolated as a solid from a suitable organic solvent by the isolation methods such as described above.

In another embodiment, the organic solvent used to isolate the oxirane compound of formula 6 is selected from the group consisting of an alcohol, a ketone, a nitrile, an ester, an ether, a polar aprotic solvents, and mixtures thereof. Specific solvents are ester solvents and more specifically ethyl acetate.

In one embodiment, a specific oxirane compound of formula 6 prepared by the process described herein is (R)-[3-nitro-4-(phenylmethoxy)phenyl]-oxirane of formula 6a (formula 6, wherein P₁ is benzyl):

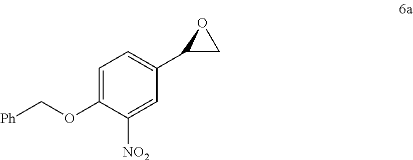

In another embodiment, the condensation reaction in step-(c) is carried out at a temperature of about 50° C. to about 150° C. for at least 2 hours, specifically at a temperature of about 70° C. to about 130° C. for about 3 hours to about 20 hours, and most specifically at a temperature of about 90° C. to about 120° C. for about 8 hours to about 12 hours.

In one embodiment, a specific compound of formula 4 prepared by the process described herein is (R,R)-α-[[[2-(4-Methoxyphenyl)-1-methylethyl](phenylmethyl)amino]methyl]-3-nitro-4-(phenylmethoxy)-benzene methanol of formula 4a (formula 4, wherein P₁ and P₂ are benzyl):

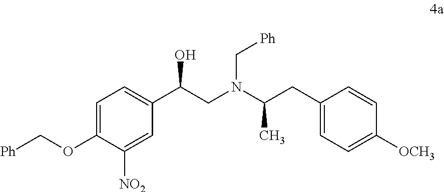

In another embodiment, the solvent used in step-(d) is selected from the group consisting of water, an alcohol, a ketone, a nitrile, an ether, a polar aprotic solvent, and mixtures thereof.

Specifically, the solvent used in step-(d) is selected from the group consisting of water, methanol, ethanol, n-propanol, isopropanol, n-butanol, isobutanol, tert-butanol, amyl alcohol, hexanol, acetone, methyl ethyl ketone, methyl isobutyl ketone, methyl tert-butyl ketone, acetonitrile, tetrahydrofuran, dioxane, diethyl ether, diisopropyl ether, monoglyme, diglyme, N,N-dimethylformamide, N,N-dimethylacetamide, dimethylsulfoxide, and mixtures thereof; and more specifically the solvent is selected from the group consisting of water, acetone, methanol, ethanol, n-propanol, isopropanol, and mixtures thereof.

In one embodiment, the reaction in step-(d) is carried out in the presence of a base. The base can be an organic or inorganic base. Specific organic bases are triethylamine, tributylamine, diisopropylethylamine, diethylamine, tert-butyl amine, N-methylmorpholine, pyridine, 4-(N,N-dimethylamino)pyridine, and mixtures thereof. In another embodiment, the base is an inorganic base selected from the group as described above. A most specific inorganic base is aqueous ammonia.

In another embodiment, the sodium dithionite in step-(d) is used in a molar ratio of about 2 to 5 moles, specifically about 3 to 5 moles, per 1 mole of the (R,R)-nitro alcohol compound of formula 4 in order to ensure a proper course of the reaction.

In one embodiment, the reaction in step-(d) is carried out at a temperature of about 0° C. to about 100° C. for at least 30 minutes, specifically at a temperature of about 20° C. to about 80° C. for about 1 hour to about 6 hours, and most specifically at a temperature of about 25° C. to about 65° C. for about 2 hours to about 5 hours.

The reaction mass containing the (R,R)-amino alcohol compound of formula 3 obtained in step-(d) may be subjected to usual work methods as described above.

In another embodiment, the (R,R)-amino alcohol compound of formula 3 formed in step-(d) is isolated as a solid from a suitable solvent by methods as described above.

In another embodiment, the solvent used to isolate the (R,R)-amino alcohol compound of formula 3 is selected from the group consisting of water, an alcohol, a ketone, a nitrile, an ester, an ether, a polar aprotic solvent, and mixtures thereof. Specifically the solvent is selected from the group consisting of water, methanol, ethanol, n-propanol, isopropanol, n-butanol, isobutanol, tert-butanol, amyl alcohol, hexanol, tetrahydrofuran, dioxane, diethyl ether, diisopropyl ether, and mixture thereof.

In one embodiment, a specific compound of formula 3 prepared by the process described herein is (R,R)-3-amino-α-[[[2-(4-methoxyphenyl)-1-methylethyl](phenylmethyl)amino]methyl]-4-(phenylmethoxy)-benzenemethanol of formula 3a (formula 3, wherein P₁ and P₂ are benzyl):

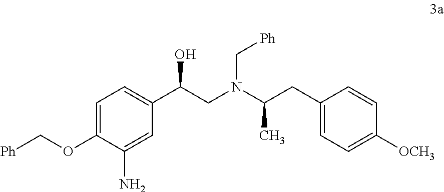

In another embodiment, the polyethylene glycol used in step-(e) is selected from the grades consisting of glycols of 300, 400, 1000, 1500, 4000, and 6000 grades, and more specifically polyethylene glycol of 400 grade.

In another embodiment, the formic acid in step-(e) is used in a molar ratio of about 2 to 5 moles, specifically about 3 to 5 moles, per 1 mole of the (R,R)-amino alcohol compound of formula 3 in order to ensure a proper course of the reaction.

In one embodiment, the reaction in step-(e) is carried out at a temperature of about 0° C. to about 110° C. for at least 1 hour, specifically at a temperature of about 20° C. to about 90° C. for about 2 hours to about 10 hours, and most specifically at a temperature of about 50° C. to about 70° C. for about 4 hours to about 8 hours.

The reaction mass containing the (R,R)-formamide compound of formula 2 obtained in step-(e) may be subjected to usual work up methods as described above.

In another embodiment, the (R,R)-formamide compound of formula 2 formed in step-(e) is isolated from a suitable solvent by methods as described above.

In one embodiment, a specific compound of formula 2 prepared by the process described herein is N-[5-[(1R)-Hydroxy-2-[[(1R)-methyl-2-(4-methoxyphenyl)ethyl](phenylmethyl)amino]ethyl]-2-(phenylmethoxy)phenyl]-formamide of formula 2a (formula 2, wherein P₁ and P₂ are benzyl):

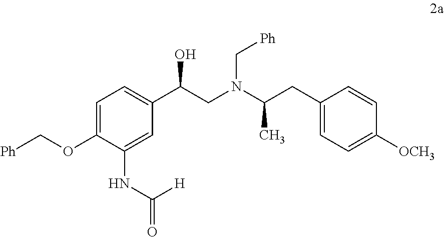

The deprotection in step-(f) is carried out by the methods known in the art. In one embodiment, the removal of the protecting groups is achieved by catalytic hydrogenation.

Exemplary hydrogenation catalysts include, but are not limited to, palladium hydroxide, palladium on carbon, platinum on carbon, platinum oxide, rhodium on carbon, and rhodium on alumina. A specific hydrogenation catalyst is palladium on carbon.

Exemplary solvents used for the hydrogenation include, but are not limited to, water, an alcohol, a ketone, an ester, a nitrile, a polar aprotic solvent, and mixtures thereof. Specifically, the solvent is selected from the group consisting of water, methanol, ethanol, isopropyl alcohol, propanol, tert-butyl alcohol, n-butanol, acetone, methyl ethyl ketone, methyl isobutyl ketone, diethyl ketone, ethyl acetate, methyl acetate, isopropyl acetate, tert-butyl methyl acetate, ethyl formate, acetonitrile, tetrahydrofuran, dimethylformamide, dimethylsulfoxide, dioxane, diethyl carbonate, and mixtures thereof; more specifically, the solvent is selected from the group consisting of water, methanol, ethanol, isopropyl alcohol, n-butanol, and mixtures thereof; and most specifically, water, methanol, n-butanol, and mixtures thereof.

The hydrogenation reaction is carried out at a temperature of below about 50° C. for at least 30 minutes, specifically at a temperature of about −25° C. to about 40° C. for about 1 hour to about 7 hours, and more specifically at a temperature of about 0° C. to about 30° C. for about 2 hours to about 5 hours.

In one embodiment, the hydrogenation catalyst is used in the ratio of about 0.05% (w/w) to 15% (w/w), specifically about 1% (w/w) to 10% (w/w), with respect to the (R,R)-formamide compound of formula 2 in order to ensure a proper course of the reaction.

The reaction mass containing the arformoterol of formula 1 obtained in step-(f) may be subjected to usual work methods as described above.

In one embodiment, the arformoterol of formula 1 formed in step-(f) is isolated as a solid from a suitable solvent by the methods as described above. Specifically, the arformoterol is isolated as a solid from a suitable solvent by evaporation or vacuum drying.

In another embodiment, the solvent used to isolate the arformoterol of formula 1 is selected from the group consisting of water, an alcohol, a ketone, a nitrile, an ester, an ether, a polar aprotic solvent, and mixtures thereof. Specifically the solvent is selected from the group consisting of water, methanol, ethanol, n-propanol, isopropanol, n-butanol, and mixture thereof.

Pharmaceutically acceptable salts of arformoterol can be prepared in high purity by using the substantially pure arformoterol obtained by the method disclosed herein, by known methods.

Exemplary pharmaceutically acceptable salts of arformoterol include, but are not limited to, hydrochloride, hydrobromide, oxalate, maleate, fumarate, mesylate, besylate, tosylate, tartrate and its stereoisomers. A most specific pharmaceutically acceptable salt of arformoterol is L-tartrate salt.

The substantially pure arformoterol or a pharmaceutically acceptable salt thereof obtained by the above process may be further dried in, for example, a Vacuum Tray Dryer, a Rotocon Vacuum Dryer, a Vacuum Paddle Dryer or a pilot plant Rota vapor, to further lower residual solvents. Drying can be carried out under reduced pressure until the residual solvent content reduces to the desired amount such as an amount that is within the limits given by the International Conference on Harmonization of Technical Requirements for Registration of Pharmaceuticals for Human Use (“ICH”) guidelines.

In one embodiment, the drying is carried out at atmospheric pressure or reduced pressures, such as below about 200 mm Hg, or below about 50 mm Hg, at temperatures such as about 35° C. to about 70° C. The drying can be carried out for any desired time period that provides the desired result, such as times about 1 to 20 hours. Drying may also be carried out for shorter or longer periods of time depending on the product specifications. Temperatures and pressures will be chosen based on the volatility of the solvent being used and the foregoing should be considered as only a general guidance. Drying can be suitably carried out in a tray dryer, vacuum oven, air oven, or using a fluidized bed drier, spin flash dryer, flash dryer, and the like. Drying equipment selection is well within the ordinary skill in the art.

The total purity of the arformoterol or a pharmaceutically acceptable salt thereof, preferably arformoterol L-tartrate salt, obtained by the process disclosed herein is of greater than about 98%, specifically greater than about 99%, more specifically greater than about 99.5%, and most specifically greater than about 99.9% as measured by HPLC.

According to another aspect, there is provided a process for preparing enantiomerically pure (R)-halohydrin compound of formula 7:

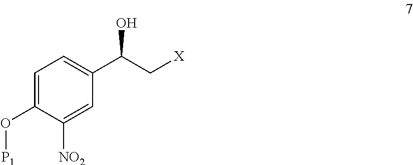

wherein ‘X’ represents a halogen atom, selected from the group consisting of F, Cl, Br and I; and ‘P₁’ is a hydroxy-protecting group; comprising reducing acetophenone compound of formula 8:

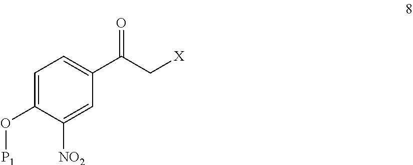

wherein ‘X’ and ‘P₁’ are as defined in formula 7;
with (−)-β-chlorodiisopinocampheylborane to produce the enantiomerically pure (R)-halohydrin compound of formula 7.

The term “enantiomerically pure (R)-halohydrin compound of formula 7” refers to the compound of formula 7 having an enantiomeric purity of greater than about 98%, specifically greater than about 99%, more specifically greater than about 99.5%, and most specifically greater than about 99.9% as measured by HPLC.

In one embodiment, the halogen atom ‘X’ in the compounds of formulae 7 and 8 is Cl or Br, and more specifically, the halogen atom is Br.

The hydroxy-protecting group ‘P₁’ in the compounds of formulae 7 and 8 is selected from the group as described above. A most specific protecting group P₁ is benzyl.

In another embodiment, the reduction is carried out in a solvent selected from the group as described above.

Specifically, the solvent is selected from the group consisting of methanol, ethanol, n-propanol, isopropanol, n-butanol, isobutanol, tert-butanol, amyl alcohol, hexanol, acetonitrile, ethyl acetate, methyl acetate, isopropyl acetate, tert-butyl methyl acetate, ethyl formate, dichloromethane, ethylene dichloride, chloroform, carbon tetrachloride, tetrahydrofuran, dioxane, diethyl ether, diisopropyl ether, monoglyme, diglyme, n-pentane, n-hexane, n-heptane, cyclohexane, toluene, xylene, N,N-dimethylformamide, N,N-dimethylacetamide, dimethylsulfoxide, and mixtures thereof; more specifically the solvent is selected from the group consisting of dichloromethane, toluene, diisopropyl ether, hexane, and mixtures thereof; and a most specific solvent is dichloromethane.

In one embodiment, the (−)-β-chlorodiisopinocampheylborane is used in a molar ratio as described above.

In another embodiment, the reaction in step-(a) is carried out at a temperature of below about 50° C. for at least 30 minutes, specifically at a temperature of about −25° C. to about 40° C. for about 1 hour to about 8 hours, and more specifically at a temperature of about 0° C. to about 30° C. for about 2 hours to about 7 hours. In another embodiment, the reaction mass may be quenched with a solution of aqueous base after completion of the reaction.

The reaction mass containing the (R)-halohydrin compound of formula 7 obtained may be subjected to usual work up and then isolated by the methods as described above.

According to another aspect, there is provided a process for preparing substantially pure (R,R)-amino alcohol compound of formula 3:

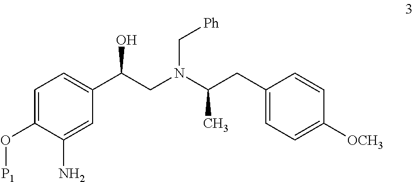

wherein ‘P₁’ is a hydroxy-protecting group, and ‘P₂’ is an amine-protecting group; comprising reducing the (R,R)-nitro alcohol compound of formula 4:

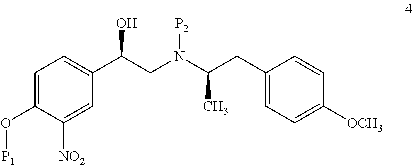

wherein the ‘P₁’ and ‘P₂’ are as defined for formula 3;
with sodium dithionite in a solvent to produce (R,R)-amino alcohol compound of formula 3.

In one embodiment, the protecting groups ‘P₁’ and ‘P₂’ in the compounds of formulae 3 and 4 are selected from the groups as described above.

The term “substantially pure (R,R)-amino alcohol compound of formula 3” refers to the (R,R)-amino alcohol compound of formula 3 having a total purity of greater than about 98%, specifically greater than about 99%, more specifically greater than about 99.5%, and most specifically greater than about 99.9% (measured by HPLC).

In one embodiment, the reaction is carried out in a solvent selected from the group as described above. Specifically, the solvent is selected from the group consisting of water, methanol, ethanol, n-propanol, isopropanol, n-butanol, isobutanol, tert-butanol, amyl alcohol, hexanol, acetone, methyl ethyl ketone, methyl isobutyl ketone, methyl tert-butyl ketone, acetonitrile, tetrahydrofuran, dioxane, diethyl ether, diisopropyl ether, monoglyme, diglyme, N,N-dimethylformamide, N,N-dimethylacetamide, dimethylsulfoxide, and mixtures thereof; and more specifically the solvent is selected from the group consisting of water, acetone, methanol, ethanol, n-propanol, isopropanol, and mixtures thereof.

In another embodiment, the reaction is carried out in the presence of a base selected from the group as described above.

In one embodiment, the sodium dithionite is used in a molar ratio as described above.

In another embodiment, the reaction is carried out at a temperature of about 0° C. to about 100° C. for at least 30 minutes, specifically at a temperature of about 20° C. to about 80° C. for about 1 hour to about 6 hours, and most specifically at a temperature of about 25° C. to about 65° C. for about 2 hours to about 5 hours.

The reaction mass containing the (R,R)-amino alcohol compound of formula 3 obtained may be subjected to usual work up and then isolated by the methods as described above.

According to another aspect, there is provided a process for preparing substantially pure (R,R)-formamide compound of formula 2:

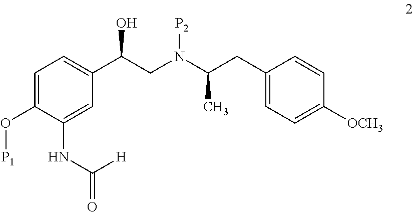

wherein ‘P₁’ is a hydroxy-protecting group, and ‘P₂’ is an amine-protecting group; comprising formylating the compound of formula 3:

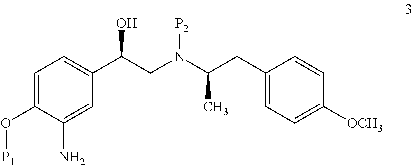

wherein the ‘P₁’ and ‘P₂’ are as defined for formula 2;
with formic acid in the presence of polyethylene glycol to produce the (R,R)-formamide compound of formula 2.

In one embodiment, the protecting groups ‘P₁’ and ‘P₂’ in the compounds of formulae 3 and 4 are selected from the groups as described above.

The term “substantially pure (R,R)-formamide compound of formula 2” refers to the (R,R)-formamide compound of formula 2 having a total purity of greater than about 98%, specifically greater than about 99%, more specifically greater than about 99.5%, and most specifically greater than about 99.9% (measured by HPLC).

In one embodiment, the polyethylene glycol used herein is selected from the grades as described above.

In another embodiment, the formic acid is used in a molar ratio as described above.

In one embodiment, the formylation reaction is carried out at a temperature of about 0° C. to about 110° C. for at least 1 hour, specifically at a temperature of about 20° C. to about 90° C. for about 2 hours to about 10 hours, and most specifically at a temperature of about 50° C. to about 70° C. for about 4 hours to about 8 hours.

The reaction mass containing the (R,R)-formamide compound of formula 2 obtained may be subjected to usual work up and then isolated by the methods as described above.

According to another aspect, there is provided a process for the preparation of arformoterol intermediate, (R)-4-methoxy-α-methyl-N-(substituted or unsubstituted phenylmethyl)benzeneethanamine of formula 5a:

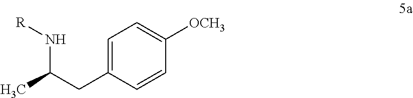

or an acid addition salt thereof, wherein ‘R’ is a protecting group selected from benzyl or substituted benzyl, comprising:
a) reacting 4-methoxyphenyl acetone of formula 9:

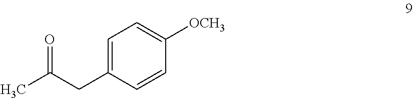

with an amine compound of formula 10:

R—NH₂ 10
wherein R is benzyl or substituted benzyl group as defined in formula 5a; in a solvent to produce a reaction mass containing an imine intermediate compound;
b) reducing the imine intermediate obtained in step-(a) with a reducing agent to provide a racemic 4-methoxy-α-methyl-N-(substituted or unsubstituted phenylmethyl)benzene ethanamine of formula 5′a:

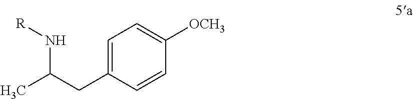

wherein R is benzyl or substituted benzyl as defined above; (or)
c) hydrogenating the imine intermediate obtained in step-(a) in the presence of platinum oxide catalyst in an ether solvent to provide the racemic benzeneethanamine compound of formula 5′a;
d) reacting the racemic benzeneethanamine compound of formula 5′a obtained step-(b) or step-(c) with L-(+)-mandelic acid in an ether solvent to produce a diastereomeric excess of L-(+)-mandelate salt of the compound of formula 5a;
e) optionally, separating the diastereomers of the L-(+)-mandelate salt of the compound of formula 5a obtained in step-(d); and
f) neutralizing the product of step-(d) or separated diastereomers of step-(e) with a base in a solvent to provide enantiomerically pure (R)-benzeneethanamine compound of formula 5a, and optionally converting the compound of formula 5a obtained into its acid addition salts thereof.

The term “enantiomerically pure benzeneethanamine compound of formula 5a” refers to the benzeneethanamine compound of formula 5a having an enantiomeric purity of greater than about 95%, specifically greater than about 98%, more specifically greater than about 99%, and most specifically greater than about 99.98% measured by HPLC.

The term “substituted benzyl” as used herein refers to an amine protecting group that contains the benzyl (or phenylmethyl) nucleus substituted with one or more substituents that do not interfere with its function as a protecting group. Exemplary substituents include, but are not limited to, C₁ to C₆-alkyl, C₁ to C₆-alkoxyl, halogen and combinations thereof.

In one embodiment, the protecting group R is benzyl.

In another embodiment, a specific benzeneethanamine compound of formula 5a prepared by the process disclosed herein is (R)-4-methoxy-α-methyl-N-(phenylmethyl)benzene ethanamine of formula 5a(i) (formula 5a, wherein R is benzyl):

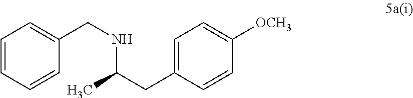

In one embodiment, the solvent used in step-(a) is selected from the group consisting of an alcohol, a ketone, a cyclic ether, an aliphatic ether, a chlorinated hydrocarbon, an ester, and mixtures thereof. Specifically, the solvent is selected from the group consisting of methanol, ethanol, isopropyl alcohol, n-butanol, tert-butanol, acetone, tetrahydrofuran, dioxane, diethyl ether, diisopropyl ether, tert-butylmethyl ether, dichloromethane, dichloroethane, ethyl acetate, isopropyl acetate, and mixtures thereof; and more specifically methanol, ethanol, isopropyl alcohol, dichloromethane, dichloroethane, tetrahydrofuran, dioxane, ethyl acetate, and mixtures thereof.

In another embodiment, the reaction in step-(a) is carried out at a temperature of below about 50° C. for at least 30 minutes, specifically at a temperature of about −25° C. to about 45° C. for about 1 hour to about 8 hours, and more specifically at a temperature of about 20° C. to about 40° C. for about 1 hour 30 minutes to about 4 hours.

In one embodiment, the reaction mass containing the imine intermediate compound formed in step-(a) is used directly in the next step to produce the racemic benzeneethanamine compound of formula 5′a.

In another embodiment, the reducing agent used in step-(b) includes, but is not limited to, a metal hydride such as sodium borohydride, sodium cyanoborohydride and sodium triacetoxyborohydride.

In one embodiment, the reduction reaction in step-(b) is carried out in an organic solvent selected from the group consisting of an alcohol, a ketone, a cyclic ether, an aliphatic ether, a chlorinated hydrocarbon, an ester, and mixtures thereof. Specifically, the organic solvent is selected from the group consisting of methanol, ethanol, isopropyl alcohol, n-butanol, tert-butanol, acetone, tetrahydrofuran, dioxane, diethyl ether, diisopropyl ether, tert-butylmethyl ether, dichloromethane, dichloroethane, ethyl acetate, isopropyl acetate, and mixtures thereof; and more specifically methanol, ethanol, isopropyl alcohol, dichloromethane, dichloroethane, tetrahydrofuran, dioxane, ethyl acetate, and mixtures thereof.

In another embodiment, the reducing agent in step-(b) is used in a molar ratio of about 0.5 to 2.6 moles, specifically, about 1.5 to 2.5 moles, with respect to the 4-methoxyphenyl acetone of formula 9.

In one embodiment, the reduction in step-(b) is carried out at a temperature of below about 50° C. for at least 30 minutes, specifically at a temperature of about −25° C. to about 40° C. for about 1 hour to about 10 hours, and more specifically at a temperature of about 15° C. to about 25° C. for about 2 hours to about 5 hours. In another embodiment, the reaction mass may be quenched with water after completion of the reaction.

The reaction mass containing the racemic benzeneethanamine compound of formula 5′a obtained in step-(b) may be subjected to usual work up and then isolated as a solid by the methods as described above. The reaction mass may be used directly in the step-(d) or the racemic benzeneethanamine compound of formula 5′a may be isolated and then used in the step-(d).

Exemplary ether solvents used in steps-(c) and (d) include, but are not limited to, cyclic ethers, aliphatic ethers, and mixtures thereof. Specifically, the ether solvent used in steps-(c) and (d) is, each independently, selected from the group consisting of tetrahydrofuran, dioxane, diethyl ether, diisopropyl ether, tert-butylmethyl ether, monoglyme, diglyme, and mixtures thereof; and more specifically tetrahydrofuran.

In one embodiment, the hydrogenation reaction in step-(c) is carried out at a temperature of below about 50° C. for at least 1 hour, specifically at a temperature of about −25° C. to about 40° C. for about 2 hours to about 15 hours, and more specifically at a temperature of about 0° C. to about 30° C. for about 5 hours to about 10 hours.

In another embodiment, the platinum oxide catalyst in step-(c) is used in a ratio of about 0.05% (w/w) to 5% (w/w), specifically, about 0.2% (w/w) to 0.6% (w/w), with respect to the 4-methoxyphenyl acetone of formula 9.

The reaction mass containing the racemic benzeneethanamine compound of formula 5′a obtained in step-(c) may be subjected to usual work up and then isolated by the methods as described above. The reaction mass may be used directly in the step-(d) or the racemic benzeneethanamine compound of formula 5′a may be isolated and then used in the step-(d).

In another embodiment, the L-(+)-mandelic acid in step-(d) is used in a molar ratio of about 0.5 to 2.0 moles, specifically, about 1.0 to 1.5 moles, per 1 mole of the racemic benzeneethanamine compound of formula 5′a.

In one embodiment, the reaction in step-(d) is carried out at a temperature of 0° C. to the reflux temperature of the solvent used for at least 30 minutes, specifically at a temperature of about 20° C. to the reflux temperature of the solvent used for about 45 minutes to about 10 hours, more specifically at a temperature of about 50° C. to the reflux temperature of the solvent used for about 1 hour to about 8 hours, and most specifically at the reflux temperature of the solvent used for about 1 hour 30 minutes to about 5 hours.

The term “diastereomeric excess” refers to the formation of a diastereomer having one configuration at chiral carbon of L-(+)-mandelic acid salt of the compound of formula 5a in excess over that having the opposite configuration. Specifically, one diastereomer is formed in above about 60% of the mixture of diastereomers over the other, and more specifically above about 80% of the mixture of diastereomers.

The L-(+)-mandelate salt of the compound of formula 5a formed may be used directly in the next step or the L-(+)-mandelate salt of the compound of formula 5a may be isolated from the reaction medium by the methods as described above and then used in the next step.

The separation of diastereomers in step-(e) is required to obtain stereomers with desired optical purity. It is well known that diastereomers differ in their properties such as solubility and then can be separated based on the differences in their properties. The separation of the diastereomers can be performed using the methods known to the person skilled in the art. These methods include chromatographic techniques and fractional crystallization, and specifically fractional crystallization.

In one embodiment, the solution of the diastereomeric mixture is subjected to fractional crystallization. The solution of the diastereomeric mixture may be a solution of the reaction mixture obtained as above or a solution prepared by dissolving the isolated diastereomeric mixture in an ether solvent. Specifically, the ether solvent is selected from the group consisting of tetrahydrofuran, dioxane, diethyl ether, diisopropyl ether, tert-butylmethyl ether, monoglyme, diglyme, and mixtures thereof; and more specifically tetrahydrofuran.

Fractional crystallization of preferentially one diastereomer from the solution of mixture of diastereomers can be performed by conventional methods such as cooling, partial removal of solvents, using anti-solvent, seeding or a combination thereof.

The fractional crystallization can be repeated until the desired chiral purity is obtained. But, usually one or two crystallizations may be sufficient.

In one embodiment, the base used in step-(f) is an organic or inorganic base selected from the group as described above.

Exemplary solvents used in step-(f) include, but are not limited to, water, an alcohol, a ketone, a cyclic ether, an aliphatic ether, a hydrocarbon, a chlorinated hydrocarbon, a nitrile, an ester, a polar aprotic solvent, and mixtures thereof. Specifically, the solvent is selected from the group consisting of water, methanol, ethanol, n-propanol, isopropanol, n-butanol, isobutanol, tert-butanol, amyl alcohol, hexanol, acetone, methyl ethyl ketone, methyl isobutyl ketone, methyl tert-butyl ketone, acetonitrile, ethyl acetate, methyl acetate, isopropyl acetate, tert-butyl methyl acetate, ethyl formate, dichloromethane, dichloroethane, chloroform, carbon tetrachloride, tetrahydrofuran, dioxane, diethyl ether, diisopropyl ether, monoglyme, diglyme, n-pentane, n-hexane, n-heptane, cyclohexane, toluene, xylene, N,N-dimethylformamide, N,N-dimethylacetamide, dimethylsulfoxide, and mixtures thereof; more specifically the solvent is selected from the group consisting of water, acetone, methanol, ethanol, n-propanol, isopropanol, n-butanol, isobutanol, tert-butanol, dichloromethane, dichloroethane, chloroform, carbon tetrachloride, tetrahydrofuran, and mixtures thereof; and most specifically the solvent is selected from the group consisting of water, methanol, ethanol, isopropanol, dichloromethane, tetrahydrofuran, and mixtures thereof.

The reaction mass containing the enantiomerically pure compound of formula 5a obtained in step-(f) may be subjected to usual work up, followed by isolation from a suitable organic solvent by methods as described above.

The use of inexpensive, non-explosive, non-hazardous, readily available and easy to handle reagents and solvents allows the process disclosed herein to be suitable for preparation of arformoterol intermediate at lab scale and in commercial scale operations.

Acid addition salts of (R)-benzeneethanamine compound of formula 5a can be prepared in high purity by using the substantially pure (R)-benzeneethanamine compound of formula 5a by the method disclosed herein, by known methods.

The acid addition salt of (R)-benzeneethanamine compound of formula 5a are derived from a therapeutically acceptable acid selected from the group consisting of hydrochloric acid, hydrobromic acid, sulfuric acid, nitric acid, phosphoric acid, acetic acid, propionic acid, oxalic acid, succinic acid, maleic acid, fumaric acid, methanesulfonic acid, benzenesulfonic acid, toluenesulfonic acid, citric acid, glutaric acid, citraconic acid, glutaconic acid, and tartaric acid. A specific acid addition salt of the (R)-benzeneethanamine compound of formula 5a is hydrochloride salt.

The term “substantially pure (R)-benzeneethanamine compound of formula 5a or an acid addition salt thereof” refers to the (R)-benzeneethanamine compound of formula 5a or an acid addition salt thereof having purity greater than about 97%, specifically greater than about 98%, and more specifically greater than about 99% measured by HPLC.

Arformoterol or a pharmaceutically acceptable acid addition salt thereof can be prepared in high purity by using the substantially pure (R)-benzeneethanamine compound of formula 5a or an acid addition salt thereof obtained by the methods disclosed herein, by known methods or by the methods described above.

According to another aspect, there is provided a process for preparing racemic 4-methoxy-α-methyl-N-(substituted or unsubstituted phenylmethyl)benzeneethanamine of formula 5′a:

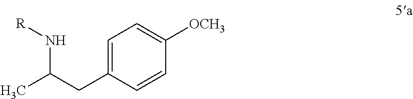

wherein ‘R’ is benzyl or substituted benzyl protecting group, comprising:
a) reacting 4-methoxyphenyl acetone of formula 9:

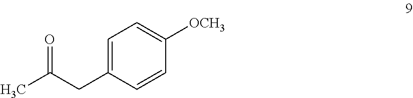

with an amine compound of formula 10:

R—NH₂ 10
wherein R is benzyl or substituted benzyl group, in a solvent to produce a reaction mass containing imine intermediate compound; and
b) reducing the imine intermediate obtained in step-(a) with a reducing agent to provide racemic benzeneethanamine compound of formula 5′a.

In one embodiment, the solvent used in step-(a) is selected from the group consisting of an alcohol, a ketone, a cyclic ether, an aliphatic ether, a chlorinated hydrocarbon, an ester, and mixtures thereof. Specifically, the solvent is selected from the group consisting of methanol, ethanol, isopropyl alcohol, n-butanol, tert-butanol, acetone, tetrahydrofuran, dioxane, diethyl ether, diisopropyl ether, tert-butylmethyl ether, dichloromethane, dichloroethane, ethyl acetate, isopropyl acetate, and mixtures thereof; and more specifically methanol, ethanol, isopropyl alcohol, dichloromethane, dichloroethane, tetrahydrofuran, dioxane, ethyl acetate, and mixtures thereof.

In another embodiment, the reaction in step-(a) is carried out at a temperature of below about 50° C. for at least 30 minutes, specifically at a temperature of about −25° C. to about 45° C. for about 1 hour to about 8 hours, and more specifically at a temperature of about 20° C. to about 40° C. for about 1 hour 30 minutes to about 4 hours.

The reducing agent used in step-(b) includes, but are not limited to, a metal hydride such as sodium borohydride, sodium cyanoborohydride and sodium triacetoxyborohydride.

In one embodiment, the reduction reaction in step-(b) is carried out in an organic solvent selected from the group as described above.

In another embodiment, the reducing agent in step-(b) is used in a molar ratio as described above.

In one embodiment, the reduction in step-(b) is carried out at a temperature of below about 50° C. for at least 30 minutes, specifically at a temperature of about −25° C. to about 40° C. for about 1 hour to about 10 hours, and more specifically at a temperature of about 15° C. to about 25° C. for about 2 hours to about 5 hours. In another embodiment, the reaction mass may be quenched with water after completion of the reaction.

The reaction mass containing the racemic benzeneethanamine compound of formula 5′a obtained in step-(b) may be subjected to usual work up and then isolated by the methods as described above.

According to another aspect, there is provided a process for preparing racemic 4-methoxy-α-methyl-N-(substituted or unsubstituted phenylmethyl)benzeneethanamine of formula 5′a:

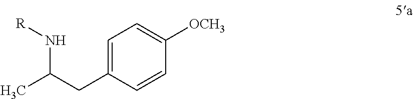

wherein ‘R’ is benzyl or substituted benzyl protecting group, comprising:
a) reacting 4-methoxyphenyl acetone of formula 9:

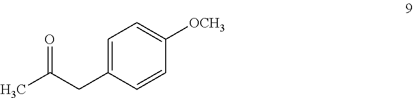

with an amine compound of formula 10:

R—NH₂ 10
wherein R is benzyl or substituted benzyl group; in a solvent to produce a reaction mass containing imine intermediate compound; and
b) hydrogenating the imine intermediate compound obtained in step-(a) in the presence of platinum oxide catalyst in an ether solvent to provide the racemic benzeneethanamine compound of formula 5′a.

The solvent used in step-(a) is selected from the group consisting of as described above.

Exemplary ether solvents used in step-(b) include, but are not limited to, a cyclic ether, an aliphatic ether, and mixtures thereof. Specifically, the ether solvent is selected from the group consisting of tetrahydrofuran, dioxane, diethyl ether, diisopropyl ether, tert-butylmethyl ether, monoglyme, diglyme, and mixtures thereof; and more specifically tetrahydrofuran.

In one embodiment, the hydrogenation reaction is carried out at a temperature of below about 50° C. for at least 1 hour, more specifically at a temperature of about −25° C. to about 40° C. for about 2 hours to about 15 hours, and more specifically at a temperature of about 0° C. to about 30° C. for about 5 hours to about 10 hours.

In another embodiment, the platinum oxide catalyst is used in the ratio as described above.

The reaction mass containing the racemic benzeneethanamine compound of formula 5′a obtained in step-(b) may be subjected to usual work and then isolated by the methods as described above.

According to another aspect, there is provided a resolution process for preparing arformoterol intermediate, (R)-4-methoxy-α-methyl-N-(substituted or unsubstituted phenylmethyl)benzeneethanamine of formula 5a:

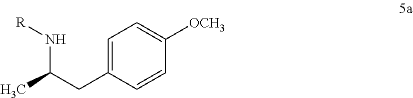

or an acid addition salt thereof, wherein ‘R’ is a benzyl or substituted benzyl group, comprising:
a) reacting the racemic benzeneethanamine compound of formula 5′a:

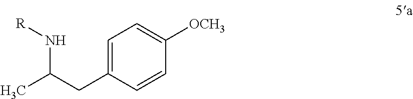

wherein ‘R’ is benzyl or substituted benzyl group;
with L-(+)-mandelic acid in an ether solvent to produce a diastereomeric excess of L-(+)-mandelate salt of the compound of formula 5a;
b) if required, separating the diastereomers of the L-(+)-mandelate salt of the compound of formula 5a obtained in step-(a); and
c) neutralizing the product of step-(a) or separated diastereomers of step-(b) with a base in a solvent to provide enantiomerically pure (R)-benzeneethanamine compound of formula 5a, and optionally converting the compound of formula 5a obtained into its acid addition salts thereof.

The term “enantiomerically pure benzeneethanamine compound of formula 5a” refers to the benzeneethanamine compound of formula 5a having enantiomeric purity greater than about 95%, specifically greater than about 98%, more specifically greater than about 99%, and most specifically greater than about 99.98% measured by HPLC.

Exemplary ether solvents used in step-(a) include, but are not limited to, a cyclic ether, an aliphatic ether, and mixtures thereof. Specifically, the ether solvent is selected from the group consisting of tetrahydrofuran, dioxane, diethyl ether, diisopropyl ether, tert-butylmethyl ether, monoglyme, diglyme, and mixtures thereof; and more specifically tetrahydrofuran.

In another embodiment, the L-(+)-mandelic acid is used in a molar ratio as described above.

In one embodiment, the reaction in step-(a) is carried out at a temperature of 0° C. to the reflux temperature of the solvent used for at least 30 minutes, specifically at a temperature of 20° C. to the reflux temperature of the solvent for about 45 minutes to about 10 hours, more specifically at a temperature of 50° C. to the reflux temperature of the solvent for about 1 hour to about 8 hours, and most specifically at the reflux temperature of the solvent for about 1 hour 30 minutes to about 5 hours.

The separation of diastereomers in step-(b) is carried out by the methods as described above.

In one embodiment, the base used in step-(c) is an organic or inorganic base selected from the group as described above.

The following examples are given for the purpose of illustrating the present disclosure and should not be considered as limitation on the scope or spirit of the disclosure.

### EXAMPLES

### Example 1

### Preparation of (R)-α-(bromomethyl)-4-phenylmethoxy-3-nitrobenzenemethanol

2-Bromo-4′-benzyloxy-3′-nitroacetophenone (10 g) was added to dichloromethane (20 ml) followed by stiffing under nitrogen atmosphere. A solution of (−)-β-chlorodiisopinocampheylborane (6.48 g) in dichloromethane (20 ml) was added slowly to the above stirred suspension for 30 minutes to 1 hour at 20-30° C. After completion of the addition process, the resulting solution was stirred for 2 hours to 4 hours at 20-30° C. The resulting mass was quenched with a solution of 5% sodium carbonate (30 ml) and the dichloromethane layer was washed with 1N sulfuric acid (30 ml) followed by 10% sodium chloride solution (30 ml). The dichloromethane layer was dried over sodium sulfate (3 g) and distilled out completely. The resulting residue was dissolved in toluene (15 ml) followed by the addition of hexane (5 ml). The precipitated product was filtered and then dried to give 9 g of (R)-α-(bromomethyl)-4-phenylmethoxy-3-nitrobenzenemethanol as a white to off white colored solid [Purity by HPLC: 98.5%; enantiomeric purity: 98% [α]^{°}_{D}=−16 to −17° (C=1, methanol)].

### Example 2

### Preparation of (R)-[3-nitro-4-(phenylmethoxy)phenyl]-oxirane

Methanol (100 ml) and tetrahydrofuran (100 ml) were added to (R)-α-(bromomethyl)-4-phenylmethoxy-3-nitrobenzenemethanol (75 g) under stirring at 20-30° C. Potassium carbonate (52.9 g) was added to the suspension at 20-30° C. and then stirred for 2 hours to 4 hours at 20-30° C. The solvents were distilled out from the resulting solution followed by quenching with water (200 ml) and the resulting mass was extracted two times with ethyl acetate (2×200 ml). Ethyl acetate was evaporated from the reaction mass under reduced pressure to provide an oily compound, followed by addition of heptane (300 ml) at 25-30° C.

The separated solid was filtered and then dried under vacuum to give 54 g of (R)-[3-nitro-4-(phenylmethoxy)phenyl]-oxirane as yellowish solid [Purity by HPLC: 98.6%; enantiomeric purity: 98.3; [α]²⁰_{D}=−9 to −11° (C=1, chloroform)].

### Example 3

### Preparation of (R,R)-α-[[[2-(4-Methoxyphenyl)-1-methylethyl](phenylmethyl)amino]methyl]-3-nitro-4-(phenylmethoxy)-benzenemethanol

(R)-[3-Nitro-4-(phenylmethoxy)phenyl]-oxirane (5 g) was added to (R)-4-methoxy-α-methyl-N-(phenylmethyl)benzene ethanamine (5.3 g) at 20-30° C., the resulting mixture was heated at 100-110° C. and then stirred for 10 hours at the same temperature. The resulting mass was cooled to 20-30° C. to give 10.3 g of (R,R)-α-[[[2-(4-methoxyphenyl)-1-methylethyl](phenylmethyl)amino]methyl]-3-nitro-4-(phenylmethoxy)-benzenemethanol [Purity by HPLC: 85%; enantiomeric purity 86%; [α]²⁰_{D}=−110 to −115° (C=1, chloroform)].

### Example 4

### Preparation of (R,R)-3-Amino-α-[[[2-(4-methoxyphenyl)-l-methylethyl](phenyl methyl)amino]methyl]-4-(phenylmethoxy)-benzenemethanol

Acetone (50 ml) was added to (R,R)-α-[[[2-(4-Methoxyphenyl)-1-methylethyl](phenylmethyl)amino]methyl]-3-nitro-4-(phenylmethoxy)-benzenemethanol (14 g) under stiffing at 20-30° C. This was followed by the addition of sodium dithionite (23 g) solution in water (50 ml) for 10 to 20 minutes at 20-60° C. The pH of the reaction mass, obtained after completion of addition process, was adjusted to 9-9.5 with aqueous ammonia (6 ml). The resulting mass was maintained at 25-60° C. for 2-4 hours. After completion of the reaction, acetone was distilled to produce oily residue. The resulting oily residue was further extracted with ethyl acetate and then concentrated to produce the residue. The residue was dissolved in methanol (28 ml) followed by the addition of diisopropyl ether (42 ml). The precipitated solid was filtered and then dried at 50-55° C. to yield 12 g of (R,R)-3-amino-α-[[[2-(4-methoxyphenyl)-1-methylethyl](phenyl methyl)amino]methyl]-4-(phenylmethoxy)-benzenemethanol as a pale yellow to brown colored solid [Purity by HPLC: 98%; enantiomeric purity: 98.99%; [α]²⁰_{D}=−120 to −125° (C=1, chloroform)].

### Example 5

### Preparation of N-[5-[(1R)-Hydroxy-2-[[(1R)-methyl-2-(4-methoxyphenyl)ethyl](phenyl methyl)amino]ethyl]-2-(phenylmethoxy)phenyl]-formamide

Polyethylene glycol-400 (90 ml) was added to (R,R)-3-amino-α-[[[2-(4-methoxyphenyl)-1-methylethyl](phenylmethyl)amino]methyl]-4-(phenylmethoxy)-benzenemethanol (45 g) under stiffing at 20-30° C. The mixture was followed by the addition of formic acid (90 ml), and the resulting mass was heated to 60-65° C. for 4-5 hours. The reaction mass was further cooled to 20-30° C. followed by quenching with water (450 ml) and adjusted the pH to 7-7.5 using 10% sodium bicarbonate solution (2 L). The reaction mass was then extracted two times with ethyl acetate (2×150 ml), the ethyl acetate layer was washed with water (450 ml) and dried over anhydrous sodium sulfate (50 g) and then distilled out the solvent to produce g of N-[5-[(1R)-hydroxy-2-[[(1R)-methyl-2-(4-methoxyphenyl)ethyl](phenylmethyl)amino]ethyl]-2-(phenyl methoxy)phenyl]-formamide [HPLC purity: 98%; enantiomeric purity: 99%; [α]²⁰_{D}=−90° to −100° (C=1, chloroform).

### Example 6

### Preparation of (R,R)-Formoterol (Arformoterol)

A solution of N-[5-[(1R)-hydroxy-2-[[(1R)-methyl-2-(4-methoxyphenyl)ethyl](phenyl methyl)amino]ethyl]-2-(phenylmethoxy)phenyl]-formamide (20 g) in methanol (200 ml) was hydrogenated in a hydrogenation flask under pressure of 1-2 Kg in the presence of Pd/C catalyst (10% Pd, 50% wet, 10 g) until the completion of the reaction. The reaction mixture was filtered through celite bed and washed with methanol (20 ml) followed by distillation of methanol on a rotavapour under reduced pressure to give 12.5 g of arformoterol base as amorphous solid [HPLC purity 98%; enantiomeric purity 98.5%; [α]²⁰_{D}=−40° (C=0.53%, chloroform).

### Example 7

### Preparation of (R,R)-Formoterol-L-tartrate (Arformoterol tartrate)

Ethanol (75.3 ml), isopropyl alcohol (38 ml), L-tartaric acid (5.66 g) and water (11.3 ml) were added to (R,R)-formoterol base (5 g) under stiffing at 20-30° C. The mixture was heated at 70-80° C. to form a clear solution. The solution was allowed to cool at 25-30° C. and maintained for 2 hours. The resulting mass was further cooled to 0-5° C. for 1 hour and the resulting product was collected by filtration. The product was dried under vacuum to provide 10 g of arformoterol tartrate as an off white powder. The tartrate salt was dissolved in 100 ml of 80% aqueous isopropyl alcohol, the resulting solution was cooled to 25-30° C. and maintained for 2 hours. The resulting mass was further cooled to 0-5° C. for 1 hour followed by filtration and drying at 50-60° C. to give 2.5 g of arformoterol tartrate [HPLC purity: 99.6%; Enantiomeric purity: 99.98%; [α]²⁰_{D}=−30° (C=0.61%, water)].

### Example 8

### Preparation of (R,R)-Formoterol-L-tartrate (Arformoterol tartrate)

Ethyl acetate (50 ml) and methanol (50 ml) were added to (R,R)-formoterol base (10 g) under stiffing at 20-30° C. This was followed by the addition of L-tartaric acid (4.36 g) and the reaction mixture was heated at 60-65° C. and maintained for 1 hour. The product was collected by hot filtration and washed with 1:1 mixture of methanol and ethyl acetate (10 ml). The wet tartrate salt was again transferred into another assembly followed by the addition of ethyl acetate (35 ml) and methanol (35 ml). The resulting mass was further heated at 60-65° C. and maintained for 1 hour. The resulting product was collected by hot filtration, washed with 1:1 mixture of methanol and ethyl acetate (10 ml) and then dried at 50-60° C. to provide 6 g of arformoterol tartrate [HPLC purity: 99.6%; Enantiomeric purity: 99.98%; [α]²⁰_{D}=30° (C=0.61%, water)].

### Example 9

### Preparation of racemic 4-Methoxy-α-methyl-N-(phenylmethyl)benzeneethanamine

4-Methoxyphenyl acetone (25 g) and dichloroethane (125 ml) were placed in a four necked flask at 25-30° C., followed by the slow addition of benzyl amine (16.3 g) and the resulting mixture was stirred for two hours at 20-40° C. The resulting imine intermediate was then reduced by adding sodium triacetoxyborohydride (32.1 g) slowly while maintaining the reaction mass temperature at below 5° C. and then stirred for 3 hours at 20-25° C. The reaction mass was quenched with water (50 ml) and then the aqueous layer was extracted with dichloromethane (25 ml). Dichloromethane was evaporated completely to yield 38.5 g of racemic 4-methoxy-α-methyl-N-(phenylmethyl)benzeneethanamine as a viscous liquid (Purity by HPLC: 85%).

### Example 10

### Preparation of 4-Methoxy-α-methyl-N-(phenylmethyl)benzeneethanamine

4-Methoxyphenyl acetone (0.25 Kg) and methanol (1.5 L) were placed in a four necked flask at 25-30° C., followed by the slow addition of benzyl amine (0.163 Kg) and the resulting mixture was stirred for 2 hours at 20-40° C. The resulting pale yellow colored turbid solution was further reduced by adding sodium cyanoborohydride (243.8 gm) slowly while maintaining the reaction mass temperature at below 5° C. and reaction mass was the stirred for 3 hours at 20-25° C. The reaction mass was quenched with water (2 L) and the resulting mass was extracted with dichloromethane (2×0.5 L). Dichloromethane was evaporated completely to yield 0.38 Kg of racemic 4-Methoxy-α-methyl-N-(phenylmethyl)benzeneethanamine as a viscous liquid (Purity by HPLC: 90%).

### Example 11

### Preparation of 4-Methoxy-α-methyl-N-(phenylmethyl)benzeneethanamine

4-Methoxyphenyl acetone (0.5 Kg), tetrahydrofuran (3.5 L) and benzyl amine (0.326 Kg) were placed in an autoclave and stirred for 2 hours at 20-40° C. Platinum oxide (0.5 g) was added to the resulting solution, 4-5 Kg pressure of hydrogen gas was applied and the reaction mass was maintained for 8 to 15 hours at 20-30° C. After completion of reaction, the reaction mass was unloaded, filtered followed by complete evaporation of tetrahydrofuran to yield 0.775 Kg of racemic 4-methoxy-α-methyl-N-(phenylmethyl)benzeneethanamine as a viscous liquid (Purity by HPLC: 92%).

### Example 12

### Preparation of (R)-4-Methoxy-α-methyl-N-(phenylmethyl)benzeneethanamine

Racemic 4-Methoxy-α-methyl-N-(phenylmethyl)benzeneethanamine (35 g) and (L)-(+)-mandelic acid (20.8 g) were heated in tetrahydrofuran (245 ml) at 50-55° C. for 1-2 hours. The resulting suspension was filtered, washed with tetrahydrofuran (70 ml) and the resulting wet product was stirred in a mixture of 8% sodium hydroxide solution (100 ml) and dichloromethane (200 ml). The dichloromethane layer was separated and dried over sodium sulphate (20 g) followed by distillation of dichloromethane under reduced pressure to yield 9 g of (R)-4-Methoxy-α-methyl-N-(phenylmethyl)benzeneethanamine as a viscous colorless liquid [Specific Optical Rotation (SOR): (−35°, C=1% in methanol); Purity by HPLC: 99.9%)].

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A process for the preparation of arformoterol of formula 1: 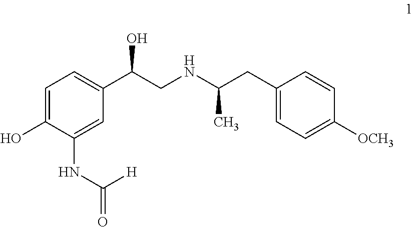
or a pharmaceutically acceptable salt thereof; comprising:
A) reducing an acetophenone compound of formula 8: 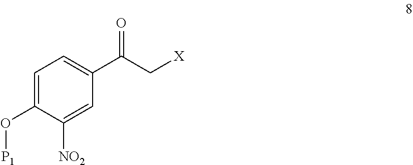
wherein ‘X’ represents a halogen atom, selected from the group consisting of F, Cl, Br and I; and ‘P₁’ is a hydroxy-protecting group;
with (−)-β-chlorodiisopinocampheylborane to produce an enantiomerically pure (R)-halohydrin compound of formula 7: 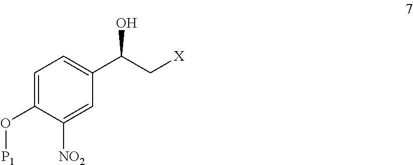
wherein ‘X’ and ‘P₁’ are as defined in formula 8;
b) treating the (R)-halohydrin compound of formula 7 with a base to produce an enantiomerically pure oxirane compound of formula 6: 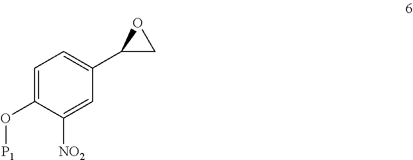
wherein ‘P₁’ is as defined in formula 8;
c) condensing the oxirane compound of formula 6 with an enantiomerically pure (R)-amine compound of formula 5: 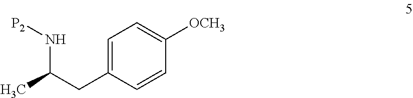
wherein ‘P₂’ is an amine-protecting group; to produce an (R,R)-nitro alcohol compound of formula 4: 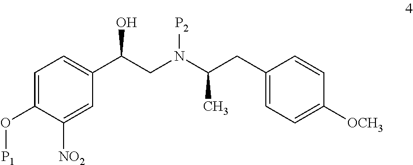
wherein the ‘P₁’ and ‘P₂’ are as defined in formulae 8 and 5;
d) reducing the (R,R)-nitro alcohol compound of formula 4 with sodium dithionite in a solvent to produce an (R,R)-amino alcohol compound of formula 3: 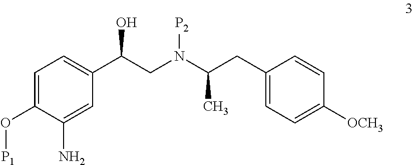
wherein the ‘P₁’ and ‘P₂’ are as defined above;
e) formylating the compound of formula 3 with formic acid in the presence of polyethylene glycol to produce an (R,R)-formamide compound of formula 2: 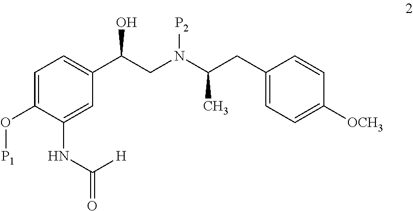
wherein the ‘P₁’ and ‘P₂’ are as defined above; and
f) deprotecting the (R,R)-formamide compound of formula 2 to produce the substantially pure arformoterol of formula 1, and optionally converting the arformoterol formed into a pharmaceutically acceptable acid addition salt thereof.

2. The process of claim 1, wherein the halogen atom ‘X’ in the compounds of formulae 7 and 8 is Br; wherein the hydroxy-protecting group ‘P₁’ in the compounds of formulae 2, 3, 4, 6, 7 and 8 is aryl- or aryloxy-substituted methyl groups; wherein the amine-protecting group ‘P₂’ in the compounds of formulae 2, 3, 4 and 5 is benzyl, or benzyl (or phenylmethyl) nucleus substituted with one or more C₁ to C₆-alkyl, C₁ to C₆-alkoxy, or halogen substituents; and wherein the pharmaceutically acceptable salt of arformoterol is a hydrochloride, a hydrobromide, an oxalate, a maleate, a fumarate, a mesylate, a besylate, a tosylate, or a tartrate salt.

3. The process of claim 2, wherein the hydroxy-protecting group ‘P₁’ is benzyl, diphenylmethyl, triphenylmethyl or benzyloxymethyl; wherein the amine-protecting group ‘P₂’ is benzyl; and wherein the pharmaceutically acceptable salt of arformoterol is L-tartrate salt.

4. The process of claim 1, wherein the reduction in step-(a) is carried out in a solvent selected from the group consisting of methanol, ethanol, n-propanol, isopropanol, n-butanol, isobutanol, tert-butanol, amyl alcohol, hexanol, acetonitrile, ethyl acetate, methyl acetate, isopropyl acetate, tert-butyl methyl acetate, ethyl formate, dichloromethane, ethylene dichloride, chloroform, carbon tetrachloride, tetrahydrofuran, dioxane, diethyl ether, diisopropyl ether, monoglyme, diglyme, n-pentane, n-hexane, n-heptane, cyclohexane, toluene, xylene, N,N-dimethylformamide, N,N-dimethylacetamide, dimethylsulfoxide, and mixtures thereof; wherein the reaction in step-(b) is carried out in a solvent selected from the group consisting of water, methanol, ethanol, n-propanol, isopropanol, n-butanol, isobutanol, tert-butanol, amyl alcohol, hexanol, acetone, methyl ethyl ketone, methyl isobutyl ketone, methyl tert-butyl ketone, acetonitrile, tetrahydrofuran, dioxane, diethyl ether, diisopropyl ether, monoglyme, diglyme, N,N-dimethylformamide, N,N-dimethylacetamide, dimethylsulfoxide, and mixtures thereof; and wherein the solvent used in step-(d) is selected from the group consisting of water, methanol, ethanol, n-propanol, isopropanol, n-butanol, isobutanol, tert-butanol, amyl alcohol, hexanol, acetone, methyl ethyl ketone, methyl isobutyl ketone, methyl tert-butyl ketone, acetonitrile, tetrahydrofuran, dioxane, diethyl ether, diisopropyl ether, monoglyme, diglyme, N,N-dimethylformamide, N,N-dimethylacetamide, dimethylsulfoxide, and mixtures thereof.

5. The process of claim 4, wherein the solvent used step-(a) is selected from the group consisting of dichloromethane, toluene, diisopropyl ether, hexane, and mixtures thereof; wherein the solvent used step-(b) is selected from the group consisting of water, tetrahydrofuran, acetone, methanol, ethanol, n-propanol, isopropanol, n-butanol, isobutanol, tert-butanol, amyl alcohol, and mixtures thereof; and wherein the solvent used step-(d) is selected from the group consisting of water, acetone, methanol, ethanol, n-propanol, isopropanol, and mixtures thereof.

6. The process of claim 1, wherein the (−)-β-chlorodiisopinocampheylborane in step-(a) is used in a molar ratio of about 0.5 to 2.5 moles per 1 mole of the acetophenone compound of formula 8; wherein the sodium dithionite in step-(d) is used in a molar ratio of about 2 to 5 moles per 1 mole of the (R,R)-nitro alcohol compound of formula 4; and wherein the formic acid in step-(e) is used in a molar ratio of about 2 to 5 moles per 1 mole of the (R,R)-amino alcohol compound of formula 3; wherein the reaction in step-(a) is carried out at a temperature of below about 50° C. for at least 30 minutes; wherein the reaction in step-(d) is carried out at a temperature of about 0° C. to about 100° C. for at least 30 minutes; and wherein the reaction in step-(e) is carried out at a temperature of about 0° C. to about 110° C. for at least 1 hour.

7. The process of claim 6, wherein the (−)-β-chlorodiisopinocampheylborane is used in a molar ratio of about 1 to 2 moles per 1 mole of the acetophenone compound of formula 8; wherein the sodium dithionite is used in a molar ratio of about 3 to 5 moles per 1 mole of the (R,R)-nitro alcohol compound of formula 4; wherein the formic acid is used in a molar ratio of about 3 to 5 moles per 1 mole of the (R,R)-amino alcohol compound of formula 3; wherein the reaction in step-(a) is carried out at a temperature of about −25° C. to about 40° C. for about 1 hour to about 8 hours; wherein the reaction in step-(d) is carried out at a temperature of about 20° C. to about 80° C. for about 1 hour to about 6 hours; and wherein the reaction in step-(e) is carried out at a temperature of about 20° C. to about 90° C. for about 2 hours to about 10 hours.

8. (canceled)

9. (canceled)

10. The process of claim 1, wherein the base used in step-(b) is an inorganic base selected from the group consisting of aqueous ammonia, sodium hydroxide, calcium hydroxide, magnesium hydroxide, potassium hydroxide, lithium hydroxide, sodium carbonate, potassium carbonate, lithium carbonate, sodium bicarbonate, potassium bicarbonate, sodium tert-butoxide, sodium isopropoxide, potassium tert-butoxide, and mixtures thereof; and wherein the reaction in step-(d) is carried out in the presence of an organic or inorganic base selected from the group consisting of triethylamine, tributylamine, diisopropylethylamine, diethylamine, tert-butyl amine, N-methylmorpholine, pyridine, 4-(N,N-dimethylamino)pyridine, aqueous ammonia, sodium hydroxide, calcium hydroxide, magnesium hydroxide, potassium hydroxide, lithium hydroxide, sodium carbonate, potassium carbonate, lithium carbonate, sodium bicarbonate, potassium bicarbonate, sodium tert-butoxide, sodium isopropoxide, potassium tert-butoxide, and mixtures thereof.

11. (canceled)

12. (canceled)

13. A process for preparing an enantiomerically pure (R)-halohydrin compound of formula 7: 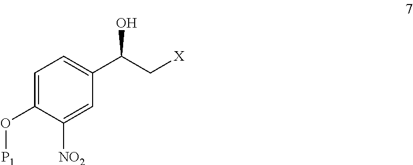
wherein ‘X’ represents a halogen atom, selected from the group consisting of F, Cl, Br and I; and ‘P₁’ is a hydroxy-protecting group; comprising reducing acetophenone compound of formula 8:
wherein ‘X’ 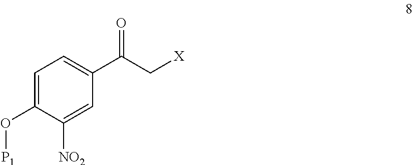
and ‘P₁’ are as defined in formula 7;
with (−)-β-chlorodiisopinocampheylborane in a solvent to produce the enantiomerically pure (R)-halohydrin compound of formula 7;
wherein the hydroxy-protecting group ‘P₁’ in the compounds of formulae 7 AND 8 is an aryl- or aryloxy-substituted methyl group.

14. (canceled)

15. The process of claim 13, wherein the hydroxy-protecting group ‘p₁’ is benzyl; and wherein the solvent is selected from the group consisting of dichloromethane, toluene, diisopropyl ether, hexane, and mixtures thereof.

16. A process for preparing a substantially pure (R,R)-amino alcohol compound of formula 3: 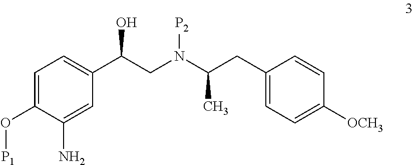
wherein ‘P₁’ is a hydroxy-protecting group, and ‘P₂’ is an amine-protecting group;
comprising reducing an (R,R)-nitro alcohol compound of formula 4: 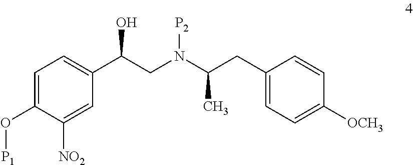
wherein the ‘P₁’ and ‘P₂’ are as defined for formula 3; with sodium dithionite in a solvent to produce the (R,R)-amino alcohol compound of formula 3.

17. The process of claim 16, wherein the hydroxy-protecting group ‘P₁’ in the compounds of formulae 3 and 4 is an aryl- or aryloxy-substituted methyl group; wherein the amine-protecting group ‘P₂’ in the compounds of formulae 3 and 4 is benzyl, or benzyl (or phenylmethyl) nucleus substituted with one or more C₁ to C₆-alkyl, C₁ to C₆-alkoxyl, halogen substituents; and wherein the solvent is selected from the group consisting of water, methanol, ethanol, n-propanol, isopropanol, n-butanol, isobutanol, tert-butanol, amyl alcohol, hexanol, acetone, methyl ethyl ketone, methyl isobutyl ketone, methyl tert-butyl ketone, acetonitrile, tetrahydrofuran, dioxane, diethyl ether, diisopropyl ether, monoglyme, diglyme, N,N-dimethylformamide, N,N-dimethylacetamide, dimethylsulfoxide, and mixtures thereof.

18. (canceled)

19. A process for preparing a substantially pure (R,R)-formamide compound of formula 2: 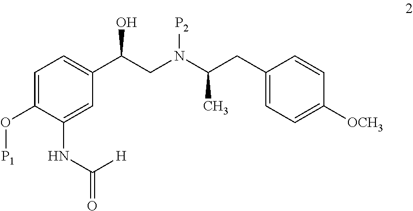
wherein ‘P₁’ is a hydroxy-protecting group, and ‘P₂’ is an amine-protecting group;
comprising formylating the compound of formula 3: 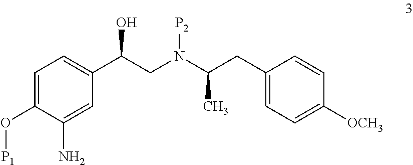
wherein the ‘P₁’ and ‘P₂’ are as defined for formula 2;
with formic acid in the presence of polyethylene glycol to produce the (R,R)-formamide compound of formula 2;
wherein the hydroxy-protecting group ‘P₁’ in the compounds of formulae 2 and 3 is an aryl- or aryloxy-substituted methyl group; and wherein the amine-protecting group ‘P₂’ in the compounds of formulae 2 and 3 is benzyl, or benzyl (or phenylmethyl) nucleus substituted with one or more C₁ to C₆-alkyl, C₁ to C₆-alkoxy, or halogen substituents.

20. (canceled)

21. A process for the preparation of arformoterol intermediate, (R)-4-methoxy-α-methyl-N-(substituted or unsubstituted phenylmethyl)benzeneethanamine of formula 5a: 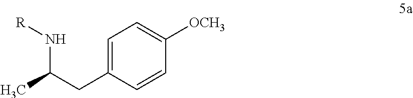
or an acid addition salt thereof, wherein ‘R’ is a protecting group selected from benzyl or substituted benzyl, comprising:
a) reacting 4-methoxyphenyl acetone of formula 9: 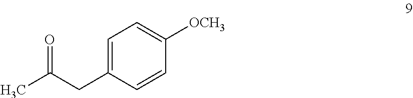
with an amine compound of formula 10:
R—NH₂ 10
wherein R is benzyl or substituted benzyl group as defined in formula 5a; in a solvent to produce a reaction mass containing an imine intermediate compound;
b) reducing the imine intermediate obtained in step-(a) with a reducing agent to provide a racemic 4-methoxy-α-methyl-N-(substituted or unsubstituted phenylmethyl)benzene ethanamine of formula 5′a: 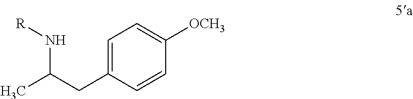
wherein R is benzyl or substituted benzyl as defined above; (or)
c) hydrogenating the imine intermediate obtained in step-(a) in the presence of platinum oxide catalyst in an ether solvent to provide the racemic benzeneethanamine compound of formula 5′a;
d) reacting the racemic benzeneethanamine compound of formula 5′a obtained step-(b) or step-(c) with L-(+)-mandelic acid in an ether solvent to produce a diastereomeric excess of L-(+)-mandelate salt of the compound of formula 5a;
e) optionally, separating the diastereomers of the L-(+)-mandelate salt of the compound of formula 5a obtained in step-(d); and
f) neutralizing the product of step-(d) or separated diastereomers of step-(e) with a base in a solvent to provide enantiomerically pure (R)-benzeneethanamine compound of formula 5a, and optionally converting the compound of formula 5a obtained into its acid addition salts thereof.

22. The process of claim 21, wherein the substituted benzyl group is a benzyl (or phenylmethyl) nucleus substituted with one or more C₁ to C₆-alkyl, C₁ to C₆- alkoxyl, or halogen substituents; wherein the solvent used in step-(a) is selected from the group consisting of methanol, ethanol, isopropyl alcohol, n-butanol, tert-butanol, acetone, tetrahydrofuran, dioxane, diethyl ether, diisopropyl ether, tert-butylmethyl ether, dichloromethane, dichloroethane, ethyl acetate, isopropyl acetate, and mixtures thereof;
wherein the solvent used in step-(b) is selected from the group consisting of methanol, ethanol, isopropyl alcohol, n-butanol, tert-butanol, acetone, tetrahydrofuran, dioxane, diethyl ether, diisopropyl ether, tert-butylmethyl ether, dichloromethane, dichloroethane, ethyl acetate, isopropyl acetate, and mixtures thereof; wherein the ether solvent used in steps-(c) and (d) is tetrahydrofuran; and wherein the solvent used in step-(f) is selected from the group consisting of water, acetone, methanol, ethanol, n-propanol, isopropanol, n-butanol, isobutanol, tert-butanol, dichloromethane, dichloroethane, chloroform, carbon tetrachloride, tetrahydrofuran, and mixtures thereof.

23. (canceled)

24. (canceled)

25. The process of claim 21, wherein the reaction in step-(a) is carried out at a temperature of below about 50° C. for at least 30 minutes; wherein the reduction in step-(b) is carried out at a temperature of below about 50° C. for at least 30 minutes; wherein the hydrogenation reaction in step-(c) is carried out at a temperature of below about 50° C. for at least 1 hour; and wherein the reaction in step-(d) is carried out at a temperature of 0° C. to the reflux temperature of the solvent used for at least 30 minutes; wherein the reducing agent used in step-(b) is a metal hydride selected from the group consisting of sodium borohydride, sodium cyanoborohydride and sodium triacetoxyborohydride; wherein the reducing agent in step-(b) is used in a molar ratio of about 0.5 to 2.6 moles with respect to the 4-methoxyphenyl acetone of formula 9; wherein the platinum oxide catalyst in step-(c) is used in a ratio of about 0.05% (w/w) to 5% (w/w) with respect to the 4-methoxyphenyl acetone of formula 9; and wherein the L-(+)-mandelic acid in step-(d) is used in a molar ratio of about 0.5 to 2.0 moles per 1 mole of the racemic benzeneethanamine compound of formula 5′a.

26. The process of claim 25, wherein the reaction in step-(a) is carried out at a temperature of at a temperature of about −25° C. to about 45° C. for about 1 hour to about 8 hours; wherein the reduction in step-(b) is carried out at a temperature of about −25° C. to about 40° C. for about 1 hour to about 10 hours; wherein the hydrogenation reaction in step-(c) is carried out at a temperature of about −25° C. to about 40° C. for about 2 hours to about 15 hours; wherein the reaction in step-(d) is carried out at a temperature of about 50° C. to the reflux temperature of the solvent used for about 1 hour to about 8 hours; wherein the reducing agent is used in a molar ratio of about 1.5 to 2.5 moles with respect to the 4-methoxyphenyl acetone of formula 9; wherein the platinum oxide catalyst is used in a ratio of about 0.2% (w/w) to 0.6% (w/w) with respect to the 4-methoxyphenyl acetone of formula 9; and wherein the L-(+)-mandelic is used in a molar ratio of about 1.0 to 1.5 moles per 1 mole of the racemic benzeneethanamine compound of formula 5′a.

27. (canceled)

28. (canceled)

29. (canceled)

30. (canceled)

31. (canceled)

32. (canceled)

33. (canceled)

34. (canceled)

35. A resolution process for preparing arformoterol intermediate, (R)-4-methoxy-α-methyl-N-(substituted or unsubstituted phenylmethyl)benzeneethanamine of formula 5a: 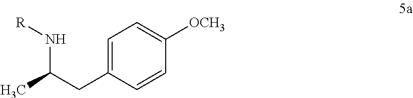
or an acid addition salt thereof, wherein ‘R’ is a benzyl or substituted benzyl group, comprising:
a) reacting the racemic benzeneethanamine compound of formula 5′a: 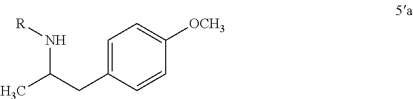
wherein ‘R’ is benzyl or substituted benzyl group;
with L-(+)-mandelic acid in an ether solvent to produce a diastereomeric excess of L-(+)-mandelate salt of the compound of formula 5a;
b) optionally, separating the diastereomers of the L-(+)-mandelate salt of the compound of formula 5a obtained in step-(a); and
c) neutralizing the product of step-(a) or separated diastereomers of step-(b) with a base in a solvent to provide enantiomerically pure (R)-benzeneethanamine compound of formula 5a, and optionally converting the compound of formula 5a obtained into its acid addition salts thereof.

36. The process of claim 35, wherein the ether solvents- used in step-(a) is selected from the group consisting of tetrahydrofuran, dioxane, diethyl ether, diisopropyl ether, tert-butylmethyl ether, monoglyme, diglyme, and mixtures thereof.
